(19) United States
(12) Patent Application Publication
(10) Pub. No.: US 2014/0171786 A1
(43) Pub. Date: Jun. 19, 2014

(54) METHOD AND SYSTEM FOR DISPLAYING INORGANIC STRUCTURE IN ULTRASOUND NON MOTION IMAGING

(71) Applicant: John Frank Stagl, Bradenton, FL (US)

(72) Inventor: John Frank Stagl, Bradenton, FL (US)

(73) Assignee: John Frank Stagl, Bradenton, FL (US)

(21) Appl. No.: 14/020,913

(22) Filed: Sep. 9, 2013

Related U.S. Application Data
(60) Provisional application No. 61/739,443, filed on Dec. 19, 2012.

(51) Int. Cl. A61B 8/08

(52) U.S. Cl. CPC A61B 8/0841
USPC 600/424

(57) ABSTRACT

The present invention provides a method and a system for identifying and coloring inorganic matter during an ultrasound scan. The identified inorganic matter is colored in a manner which enables a medical practitioner to differentiate it from the color of organic matter. The method and system include determining a tissue density threshold, obtaining an ultrasound image of the region of the body of the organism and identifying a first set of sub regions of the ultrasound image and a second set of sub regions of the ultrasound image based on the tissue density threshold. Furthermore, the method and system include coloring the second set of sub regions in the ultrasound image of the ultrasound scan. The tissue density threshold is a tissue density of a region of a body. The first set of regions pertains to organic matter and the second set of regions pertains to the inorganic matter.

### FIELD OF THE INVENTION

The present invention relates to ultrasonic imaging equipment, and more particularly, relates to methods and systems for displaying inorganic structure in the ultrasonic imaging equipment.

### BACKGROUND OF THE INVENTION

Ultrasound is a non-intrusive diagnostic technique for imaging many of the internal structures of living organisms. In recent years, the quality of ultrasound equipment has been improved and the equipment is now capable of producing images of outstanding detail and clarity. Ultrasound produces dynamic views which are useful in examining concealed structures which are subject to changes in shape or position, such as the human heart, human fetuses, the circulatory system and reproductive and internal organs.

Ultrasound can be used over the entire human body and has certain advantages over other modalities, including, among others: the ability to locate and characterize medical problems; lower cost compared to modalities such as MRI and CT; real time operation; and, the lack of ionizing radiation with the known associated health risks.

Ultrasound imaging systems transmit sound waves of very high frequency into the patient's body and the echoes scattered from structures in the patient's body are processed to create and display images and information related to these structures. Ultrasound imaging can be applied to various regions or organs in the body. For example, a breast ultrasound procedure involves the placement of an ultrasound transducer over a region of interest of the breast, with the radiologist or other medical professional (the “user”) viewing a real-time ultrasound image output on a display. The ultrasound machine monitor usually displays relevant text and/or graphical information next to the ultrasound image for simultaneous viewing by the user.

Nowadays, two dimensional (“2D”) ultrasound images, representing a slice through a region of interest of the patient's body, is the most common technique used for diagnosis. However, the two dimensional ultrasound images of living tissue and inorganic materials within the tissue are only capable of being displayed in grey scale. Examples of the inorganic matter can include but may not be limited to needles, catheters, and laser fibers. This gray scale image makes it difficult for an examiner to differentiate between organic from inorganic matter within tissue. In conventional approaches, since the inorganic matter is denser than most organic tissue, dense structures are displayed as white. However, in this approach, an inexperienced practitioner can confuse the white appearing tissues with the dense structure of the inorganic matter.

In light of the above discussion, there is a need for a method and a system that will overcome the above stated drawbacks.

### SUMMARY OF THE INVENTION

This Summary is provided to introduce a selection of concepts in a simplified form that are further described below in the Detailed Description. This Summary is not intended to identify key or essential features of the invention, nor is it intended to be used as an aid in determining the scope of the invention.

The present invention provides a method for identifying and coloring an inorganic matter during an ultrasound scan. The identified inorganic matter is colored in a manner which enables a medical practitioner to differentiate it from the color of organic matter. The method includes determining a tissue density threshold. Further, the method includes obtaining an ultrasound image of the region of the body of the organism and identifying a first set of sub regions of the ultrasound image and a second set of sub regions of the ultrasound image based on the tissue density threshold. Furthermore, the method includes highlighting the second set of sub regions in the ultrasound image of the ultrasound scan by coloring the second set of sub regions. The tissue density threshold is a tissue density of a region of a body of the organism. The first set of sub regions pertains to organic matter and the second set of sub regions pertains to the inorganic matter.

In an embodiment of the present invention, determining the tissue density threshold includes receiving a sample ultrasound image of the body of the organism and calculating the tissue density threshold using the sample ultrasound image.

In an embodiment of the present invention, the method includes generating an alert based on a pre-defined condition. In an embodiment of the present invention, the method includes receiving a sub region information of a selected sub region in the ultrasound image, calculating density of the selected sub region of the ultrasound image, identifying at least one sub region based on the calculated density of the selected region, and highlighting the at least one sub region.

In an embodiment of the present invention, highlighting the identified second set of sub regions includes drawing a boundary around each sub region of identified second set of sub regions. In an embodiment of the present invention, highlighting the identified second set of sub regions includes coloring the identified second set of regions based on a color mode. In an embodiment, the identified second set of regions is colored. In an embodiment of the present invention, the method includes adjusting spectrum and intensity of highlighted second set of sub regions.

In another aspect of the present invention, a method for highlighting inorganic matter by coloring the inorganic matter during an ultrasound scan is provided. The identified inorganic matter is colored in a manner which enables a medical practitioner to differentiate it from the color of organic matter. The method includes determining a tissue density threshold by using a sample ultrasound image of a region of a body, capturing an ultrasound image of the region of the body of the organism, identifying a first set of sub regions of the ultrasound image and a second set of sub regions of the ultrasound image based on tissue density threshold and highlighting the second set of sub regions based on a color mode in the ultrasound image of the ultrasound scan. The tissue density threshold is a tissue density of the region of the body of the organism. The first set of sub regions pertains to organic matter and the second set of sub regions pertains to the inorganic matter.

In an embodiment of the present invention, determining the tissue density threshold comprises receiving a threshold value and setting the tissue density threshold in accordance with the threshold value. In an embodiment of the present invention, the method includes generating an alert based on a pre-defined condition. In an embodiment of the present invention, the method includes receiving a sub region information of a selected sub region in the ultrasound image, calculating density of the selected sub region of the ultrasound image, identifying at least one sub region based on the calculated density of the selected region, and highlighting the at least one sub region. In an embodiment of the present invention, highlighting the identified second set of sub regions includes drawing a boundary around each sub region of identified second set of sub regions.

In another aspect of the present invention, a computer system for identifying and coloring an inorganic matter during an ultrasound scan in an ultrasound imaging apparatus is provided. The identified inorganic matter is colored in a manner which enables a medical practitioner to differentiate it from the color of organic matter. The system includes one or more processors and a non-transitory memory containing instructions that, when executed by the one or more processors, causes the one or more processors to perform a set of steps. The set of steps include determining a tissue density threshold by a determination module of the one or more processors, obtaining an ultrasound image of the region of the body of the organism by a receiving module of the one or more processors, identifying a first set of sub regions of the ultrasound image and a second set of sub regions of the ultrasound image based on the tissue density threshold by an identification module of the one or more processors, and highlighting the second set of sub regions based on a color mode in the ultrasound image of the ultrasound scan by a highlighting module of the one or more processors. The tissue density threshold is a tissue density of a region of a body of the organism. The first set of sub regions pertains to organic matter and the second set of sub regions pertains to the inorganic matter.

In an embodiment of the present invention, the determination module of the one or more processors is further adapted to receive a sample ultrasound image of the region of the organism and calculate the tissue density threshold using the sample ultrasound image. In an embodiment of the present invention, the determination module of the one or more processors is further adapted to receive a threshold value and setting the tissue density threshold in accordance with the threshold value. In an embodiment of the present invention, the one or more processors are further configured to execute the steps of receiving a sub region information of a selected sub region in the ultrasound image, calculating density of the selected sub region of the ultrasound image, identifying at least one sub region based on the calculated density of the selected region and highlighting the at least one sub region. In an embodiment of the present invention, the highlighting module of the one or more processors is adapted to draw a boundary around each sub region of identified second set of sub regions.

Other aspects and example embodiments are provided in the drawings and the detailed description that follows.

### BRIEF DESCRIPTION OF THE DRAWINGS

For a more complete understanding of example embodiments of the present technology, reference is now made to the following descriptions taken in connection with the accompanying drawings in which:

FIG. 1 illustrates a system for identifying and coloring inorganic matter during an ultrasound scan, in accordance with various embodiments of the present invention;

FIG. 2 depicts various states for performing ultrasound imaging on a patient by a medical practitioner using an ultrasound machine, in accordance with various embodiments of the present invention;

FIG. 3 illustrates a flowchart for identifying and coloring inorganic matter during the ultrasound scan using one or more processors, in accordance with an embodiment of the present invention;

FIG. 4 illustrates a flowchart for identifying and coloring inorganic matter during the ultrasound scan using the one or more processors, in accordance with another embodiment of the present invention; and

FIG. 5 illustrates a block diagram of the ultrasound machine, in accordance with another embodiment of the present invention.

### DETAILED DESCRIPTION OF THE INVENTION

In the following description, for purposes of explanation, numerous specific details are set forth in order to provide a thorough understanding of the present technology. It will be apparent, however, to one skilled in the art that the present technology can be practiced without these specific details. In other instances, structures and devices are shown in block diagram form only in order to avoid obscuring the present technology.

Reference in this specification to “one embodiment” or “an embodiment” means that a particular feature, structure, or characteristic described in connection with the embodiment is included in at least one embodiment of the present technology. The appearance of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

Moreover, although the following description contains many specifics for the purposes of illustration, anyone skilled in the art will appreciate that many variations and/or alterations to said details are within the scope of the present technology. Similarly, although many of the features of the present technology are described in terms of each other, or in conjunction with each other, one skilled in the art will appreciate that many of these features can be provided independently of other features. Accordingly, this description of the present technology is set forth without any loss of generality to, and without imposing limitations upon, the present technology.

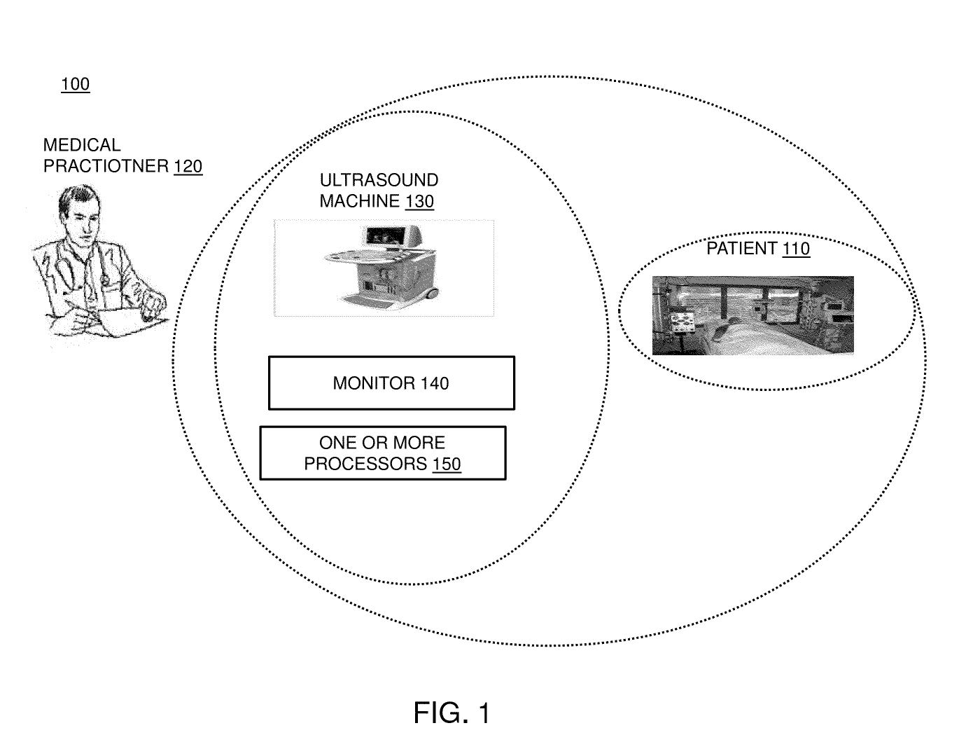

FIG. 1 illustrates a system 100 for identifying and coloring inorganic matter during an ultrasound scan, in accordance with various embodiments of the present invention. The system 100 includes a patient 110 and a medical practitioner 120. The patient 110 visits the medical practitioner 120 for certain health related issues. Examples of the health related issues may include but not limited to prostate problem, bone fractures, heart problems and cardio vascular problems. The medical practitioner 120 uses an ultrasound machine 130, which produces ultrasound images. These images enable the medical practitioner 120 to understand the patient 110 anatomy in a better way and provide better treatment to the patient 130.

The ultrasound machine 130 includes a monitor 140. The medical practitioner 120 uses the monitor 140 to see the images produced by the ultrasound machine 130. In an embodiment of the present invention, the monitor 140 can display images in grey scale. In another embodiment of the present invention, the monitor 140 can display the images in colored format. In yet another embodiment of the present invention, the monitor 140 can display a combination of color and grey scale in the same image. In an embodiment of the present invention, a decision to add color to a part of the image or to keep the image in grey scale depends on different factors (explained later in the patent application). In an embodiment, the ultrasound machine 130 includes one or more processors 150. The one or more processors 150 distinguish inorganic matter from organic matter.

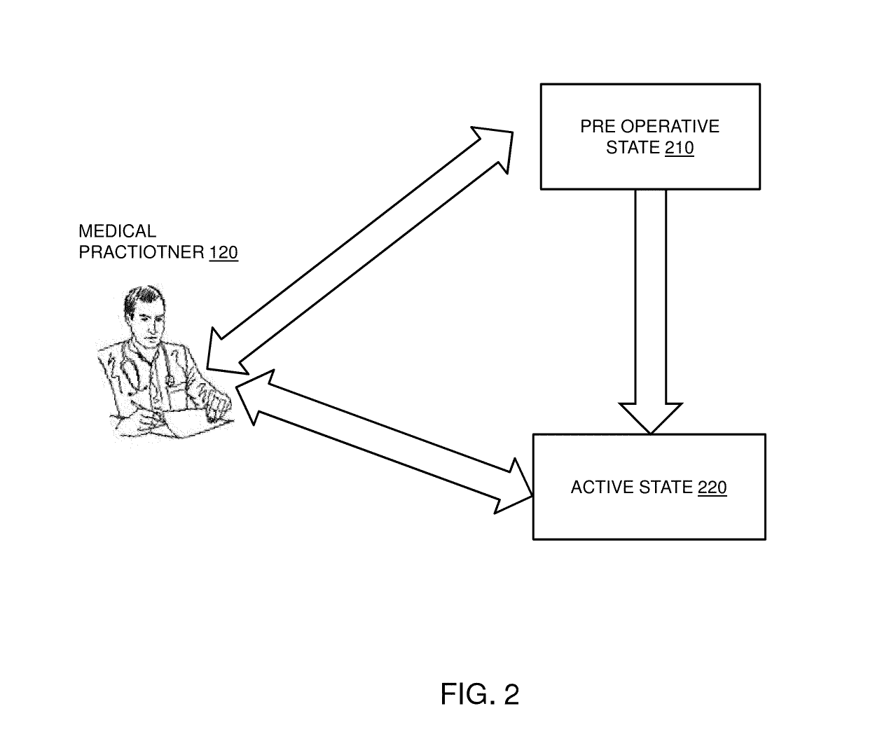

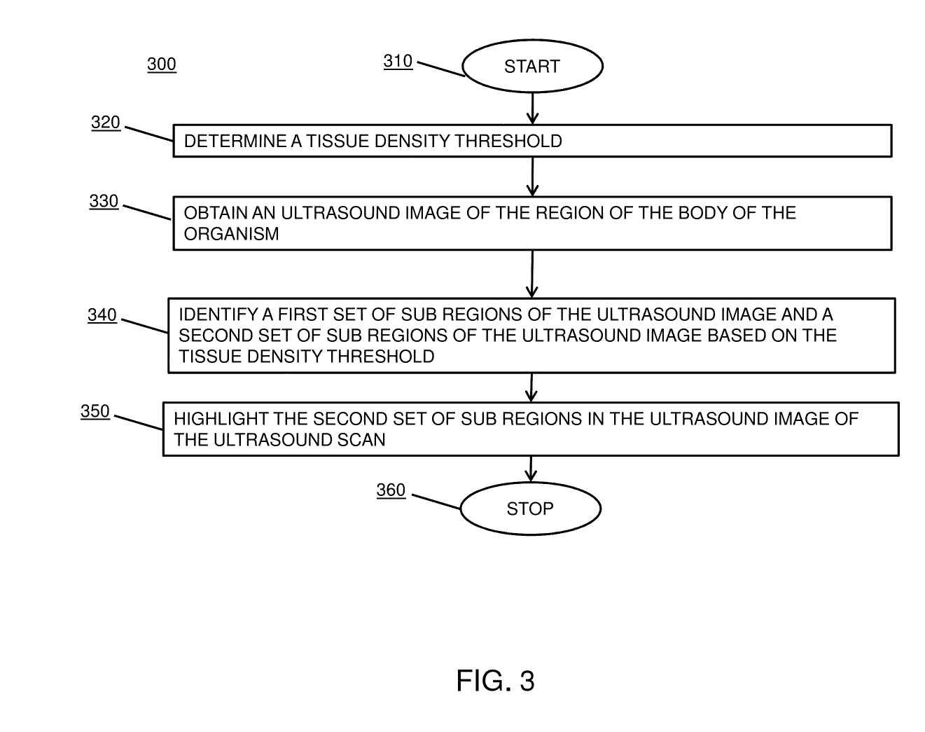

FIG. 2 depicts various states for performing ultrasound imaging on the patient 110 by the medical practitioner 120 using the ultrasound machine 130, in accordance with various embodiments of the present invention. The various states include a pre-operative state 210 and an active state 220. In the pre-operative state 210, the medical practitioner 120 switches on the ultrasound machine 130 and performs ultrasound scan on the patient 110 without inserting the inorganic matter in the body of the patient 110. The ultrasound machine 130 sets some pre-defined parameters for displaying the ultrasound image in the active state 220 for the particular patient 110 when the inorganic material is inserted into the body of the patient 110. Examples of the pre-defined parameters include but may not be limited to density of tissues of the patient 110. In an embodiment of the present invention, a range of the pre-defined parameters which will differentiate the tissues when the inorganic material is inserted in the body of the patient 110, is set for the patient 110 in the ultrasound machine 130. The range of the pre-defined parameters depends on different factors which may include but may not be limited to age of the patient 110, sex of the patient 110, weight of the patient 110, and other physio-chemical attributes of the patient 110. Once the range of the pre-defined parameters (say range for the density of the tissues) are set and stored for the patient 110, the inorganic material are inserted in the body of the patient 110 for performing the ultrasound scan. Now, in this the active state 220, the tissues inside the body of the patient 110 are displayed in grayscale and the inorganic matter is assigned a color to distinguish the inorganic matter from the tissues present inside the body. In an embodiment of the present invention, the tissues inside the body and the inorganic matter are assigned a different color based on the density. In an embodiment of the present invention, the image shown on the monitor 140 of the ultrasound machine 130 differentiates the inorganic matter (by showing it in a color mode) with the tissues of the parts of the body of the patient 110 (by showing them in grayscale mode). In an embodiment, an administrator of the ultrasound machine 130 can change the color, which is used to highlight the organic matter. In addition, the inorganic material may already be present in the body of the patient 110. The various states for this embodiment have been explained in conjunction with the description of FIG. 3.

It may be noted that while FIG. 2 shows the two states 210 and 220, those skilled in the art would appreciate that there can be a multiplicity of intermediate states between the above-mentioned states. In addition, the names the two states 210 and 220 as pre active state and active state can be named differently without having an impact on the function performed by the two states 210 and 220.

FIG. 3 illustrates a flowchart 300 for identifying and coloring inorganic matter during an ultrasound scan using the one or more processors 150, in accordance with various embodiments of the present invention.

The flowchart 300 initiates at step 310. At step 320, the one or more processors 150 determine a tissue density threshold. The tissue density threshold is a range of densities of tissues of an organism. The range of densities is determined by measuring the density of various types of tissues of the organism. In an embodiment, the one or more processors 150 determine the tissue density threshold by receiving a sample ultrasound image of the body of the organism. On basis of the sample ultrasound image, the one or more processors 150 calculate the tissue density threshold. In another embodiment, the one or more processors 150 receive value of the tissue density threshold from the medical practitioner 120. In yet another embodiment, an operator feeds the value of the tissue density threshold in the one or more processors 150 during the installation of the ultrasound machine 130. In yet another embodiment, the one or more processors 150 obtain the value of the tissue density threshold from a server. The server is pre loaded with medical history of the patient 110. The medical history of the patient 110 includes the value of the tissue density threshold calculated during the registration of the patient 110.

Although the present invention provides three embodiments in relation to determining the tissue density threshold; one skilled in the art that the present invention can use other existing techniques to determine the tissue density threshold. It may be noted that the term patient 130 and the organism is used interchangeably in the present application.

At step 330, the one or more processors 150 obtain an ultrasound image of the region of the body of the patient 130. In an embodiment, the one or more processors 150 receive ultrasound image from the ultrasound machine 130. The ultrasound machine 130 scans the region of the body of the organism using a probe. On completion of the scan, the ultrasound machine 130 provides the ultrasound image to the one or more processors 150. In another embodiment, the one or more processors 150 scan the region of the body of the patient 130 using a scanner. Examples of scanner include but may not be limited to an X-ray machine and a CT scan machine.

At step 340, the one or more processors 150 identify a first set of sub regions of the ultrasound image and a second set of sub regions of the ultrasound image. The one or more processors 150 use the density of regions for the identification. The one or more processors 150 classify a region of the ultrasound image as the first set when the density of the region is below the tissue density threshold. The one or more processors 150 classify a region of the ultrasound image as the second set when the density of the region is above the tissue density threshold.

At step 350, the one or more processors 150 highlight the second set of sub regions of the ultrasound image. In an embodiment, the one or more processors 150 highlight the second set of sub regions of the ultrasound image by drawing a boundary around each sub region from the second set of sub regions. In an embodiment, the one or more processors 150 draw a boundary around each of the sub region from the identified sub regions by selecting a set of external points of each of the sub region.

In another embodiment, the one or more processors 150 highlight the second set of sub regions of the ultrasound image by coloring the second set of sub regions on the basis of a color scheme. In an embodiment, the color scheme is pre set during manufacture. In another embodiment, the medical practitioner 120 sets the color scheme according to circumstances. In yet another embodiment, the color scheme is determined based on the region of the body of the organism being scanned.

In an embodiment, the one or more processors 150 adjust the spectrum and intensity of the highlighted second set of sub regions. In an embodiment, the one or more processors 150 receive input from the medical practitioner 120. The input includes the amount by which the spectrum and intensity need to be adjusted. The one or more processors 150 adjust the spectrum and intensity of the highlighted second set of sub regions accordingly. In another embodiment, the one or more processors 150 receive input from a photo sensor installed on the ultrasound machine 130. In an embodiment, the photo sensor is proximal to the display. Based on the input from the photo sensor, the one or more processors 150 adjust the spectrum and intensity of the second set of sub regions.

In an embodiment, the medical practitioner 120 is able to select a sub region using a pointing device. In another embodiment, the medical practitioner 120 is able to select the sub region using a touch pad. The one or more processors 150 identify the density of the selected sub region. Further, the one or more processors 150 determine sub regions of the ultrasound image with same density as that of the selected sub region. Accordingly, the one or more processors 150 highlight the determined sub regions.

Although the present invention provides two embodiments for selecting the sub region; one skilled in the art would appreciate that selecting the sub region can be performed by technologies presently known in the art. one or more processors

In an embodiment, the one or more processors 150 alert the medical practitioner 120 based on a pre-defined condition. In an embodiment, the one or more processors 150 alert the medical practitioner 120 by generating and transmitting an audio signal. An example of audio signal is a high pitch siren. In another embodiment, the one or more processors 150 alert the medical practitioner 120 using a visual alert. An example of visual alert is flashing a warning light. In an embodiment, the pre-defined condition is the detection of a substance corresponding to a list of substances available with the one or more processors 150. In an example, the list of substances includes hazardous and toxic substances. The one or more processors 150 alert the medical practitioner 120 to indicate danger to the patient 110. In another example, the list of substances includes implantable medical devices. The one or more processors 150 alert the medical practitioner 120 to indicate the presence of one or more implantable medical devices. In another embodiment, the pre-defined condition is the intensity of the highlighted sub region being below a level suitable for optimal visualization. Therefore, the one or more processors 150 raise an alert. The flowchart 300 terminates at step 360.

Although the present invention provides the six steps 310-360 to display and highlight the inorganic matter in the body of a patient 110; one skilled in the art can appreciate that there can be a multiplicity of intermediate steps in between the above mentioned six steps 310-360.

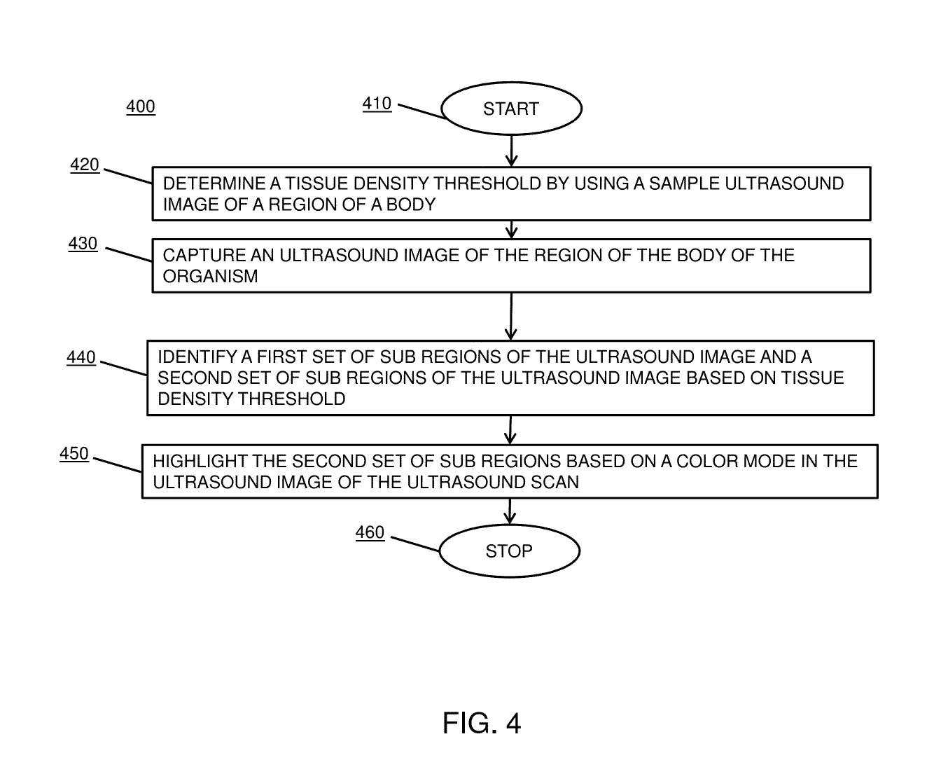

FIG. 4 illustrates a flowchart 400 for identifying and coloring inorganic matter during an ultrasound scan using the one or more processors 150, in accordance with various embodiments of the present invention. It may be noted that to explain the flow chart 400, reference to the various steps of the flow chart will be made. In addition, references to the system elements of FIG. 1 and FIG. 2 will be made. In addition, it may be noted that the flow chart 400 is explained from a different aspect of the invention to the flow chart 300 explained in the FIG. 3.

At step 410, the flow chart 400 initiates. At step 420, the one or more processors 150 determine a tissue density threshold by using a sample ultrasound image of a region of a body. At step 430, the one or more processors 150 capture the ultrasound image of the region of the body of the patient 110. At step 440, the one or more processors 150 identifies the first set of sub regions of the ultrasound image and the second set of sub regions of the ultrasound image based on tissue density threshold. At step 450, the one or more processors 150 highlights the second set of sub regions based on a color mode in the ultrasound image of the ultrasound scan. The tissue density threshold is a tissue density of the region of the body of the organism. The first set of sub regions pertains to organic matter and the second set of sub regions pertains to the inorganic matter.

In an embodiment of the present invention, determining the tissue density threshold comprises receiving a threshold value and setting the tissue density threshold in accordance with the threshold value. In an embodiment of the present invention, the method includes generating an alert based on a pre-defined condition. In an embodiment of the present invention, the method includes receiving a sub region information of a selected sub region in the ultrasound image, calculating density of the selected sub region of the ultrasound image, identifying at least one sub region based on the calculated density of the selected region, and highlighting the at least one sub region. In an embodiment of the present invention, highlighting the identified second set of sub regions includes drawing a boundary around each sub region of identified second set of sub regions. At step 460, the flowchart 400 terminates.

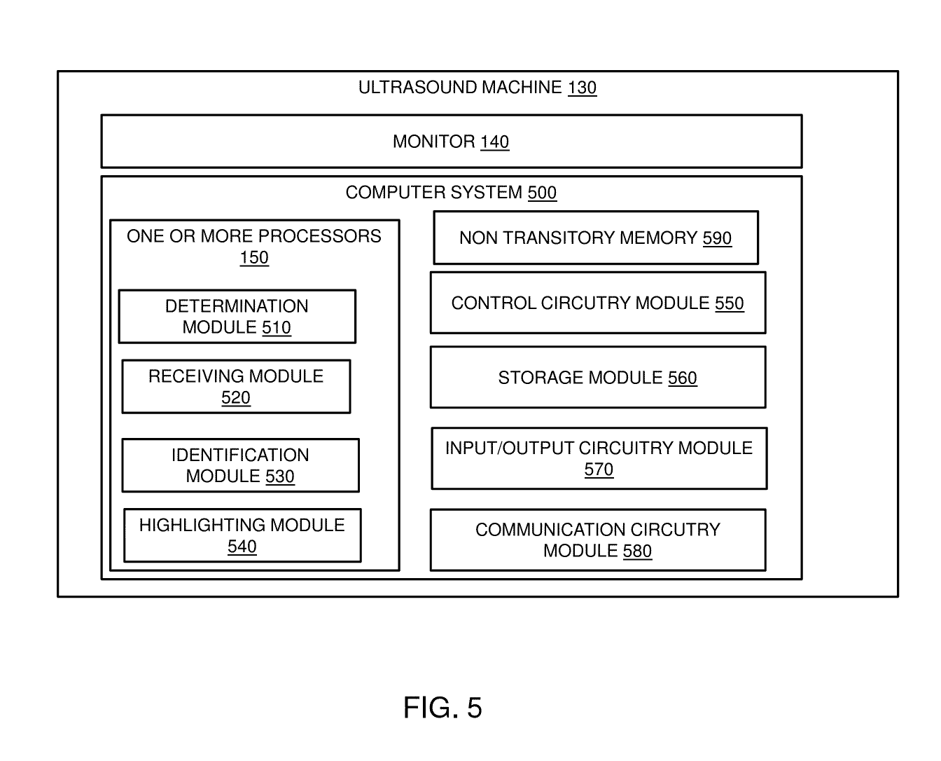

FIG. 5 illustrates a block diagram of the ultra sound machine 130, in accordance with various embodiments of the present invention. It may be noted that the block diagram 500 more or less number of system components which performs the above stated method steps. The ultra sound machine 130 includes the monitor 140 and a computer system 500. The computer system 500 includes the one or more processors 150, a control circuitry module 550, a storage module 560, a non transitory memory 590, an input output circuitry 570 and a communication circuitry 580.

In an embodiment of the present invention, the one or more processors 150 include a determination module 510, a receiving module 520, an identification module 530, and a highlighting module 540. It may be noted that the one or more processors 150 include more or less number of system components which performs the above stated method steps.

The one or more processors 150 and the non-transitory memory 590 contains instructions that, when executed by the one or more processors 150, causes the one or more processors 150 to perform a set of steps. In an embodiment, the determination module 510 determines a tissue density threshold. The receiving module 520 obtains the ultrasound image of the region of the body of the patient 110. The identification module 530 identifies a first set of sub regions of the ultrasound image and a second set of sub regions of the ultrasound image based on the tissue density threshold. The highlighting module 540 highlights the second set of sub regions based on a color mode in the ultrasound image of the ultrasound scan. Tissue density threshold is a tissue density of a region of a body of the organism. The first set of sub regions pertains to organic matter and the second set of sub regions pertains to the inorganic matter.

In an embodiment of the present invention, the determination module 510 of the one or more processors 150 is further adapted to receive a sample ultrasound image of the region of the organism and calculate the tissue density threshold using the sample ultrasound image. In an embodiment of the present invention, the determination module 510 of the one or more processors 150 is further adapted to receive a threshold value and setting the tissue density threshold in accordance with the threshold value. In an embodiment of the present invention, the one or more processors 150 are further configured to execute the steps of receiving a sub region information of a selected sub region in the ultrasound image, calculating density of the selected sub region of the ultrasound image, identifying at least one sub region based on the calculated density of the selected region and highlighting the at least one sub region. In an embodiment of the present invention, the highlighting module 540 of the one or more processors is adapted to draw a boundary around each sub region of identified second set of sub regions.

From the perspective of this invention, the control circuitry 550 includes any processing circuitry or processor operative to control the operations and performance of the computer system 500. For example, the control circuitry 550 may be used to run operating system applications, firmware applications, media playback applications, media editing applications, or any other application. In an embodiment, the control circuitry 550 drives a display and process inputs received from a user interface.

From the perspective of this invention, the storage 560 includes one or more storage mediums including a hard-drive, solid state drive, flash memory, permanent memory such as ROM, any other suitable type of storage component, or any combination thereof. The Storage 560 may store, for example, media data (e.g., music and video files), application data (e.g., for implementing functions on the computer system 500).

From the perspective of this invention, the I/O circuitry 570 may be operative to convert (and encode/decode, if necessary) analog signals and other signals into digital data. In an embodiment, the I/O circuitry 570 may also convert digital data into any other type of signal, and vice-versa. For example, the I/O circuitry 570 may receive and convert physical contact inputs (e.g., from a multi-touch screen), physical movements (e.g., from a mouse or sensor), analog audio signals (e.g., from a microphone), or any other input. The digital data may be provided to and received from the control circuitry 550, the storage 560, or any other component of the computer system 500.

It may be noted that the I/O circuitry 570 is illustrated in FIG. 5 as a single component of the computer system 500; however those skilled in the art would appreciate that several instances of the I/O circuitry 570 may be included in the computer system 500.

The computer system 500 may include any suitable interface or component for allowing a user to provide inputs to the I/O circuitry 570. The computer system 500 may include any suitable input mechanism. Examples of the input mechanism include but may not be limited to a button, keypad, dial, a click wheel, and a touch screen. In an embodiment, the computer system 500 may include a capacitive sensing mechanism, or a multi-touch capacitive sensing mechanism.

In an embodiment, the computer system 500 may include specialized output circuitry associated with output devices such as, for example, one or more audio outputs. The audio output may include one or more speakers built into the computer system 500, or an audio component that may be remotely coupled to the computer system 500.

The one or more speakers can be mono speakers, stereo speakers, or a combination of both. The audio component can be a headset, headphones or ear buds that may be coupled to communications device with a wire or wirelessly.

In an embodiment, the I/O circuitry 570 may include display circuitry for providing a display visible to the user. For example, the display circuitry may include a screen (e.g., an LCD screen) that is incorporated in the computer system 500.

The display circuitry may include a movable display or a projecting system for providing a display of content on a surface remote from the computer system 500 (e.g., a video projector). In an embodiment, the display circuitry may include a coder/decoder to convert digital media data into analog signals. For example, the display circuitry may include video Codecs, audio Codecs, or any other suitable type of Codec.

The display circuitry may include display driver circuitry, circuitry for driving display drivers, or both. The display circuitry may be operative to display content. The display content can include media playback information, application screens for applications implemented on the electronic device, information regarding ongoing communications operations, information regarding incoming communications requests, or device operation screens under the direction of the control circuitry 550. Alternatively, the display circuitry may be operative to provide instructions to a remote display.

From the prospective of this invention, the communications circuitry 580 may include any suitable communications circuitry operative to connect to a communications network and to transmit communications (e.g., voice or data) from the computer system 500 to other devices within the communications network. The communications circuitry 580 may be operative to interface with the communications network using any suitable communications protocol. Examples of the communications protocol include but may not be limited to Wi-Fi, Bluetooth.RTM, radio frequency systems, infrared, LTE, GSM, GSM plus EDGE, CDMA, and quadband.

In an embodiment, the same instance of the communications circuitry 580 may be operative to provide for communications over several communications networks. In an embodiment, the computer system 500 may be coupled a host device for data transfers, synching the communications device, software or firmware updates, providing performance information to a remote source (e.g., providing riding characteristics to a remote server) or performing any other suitable operation that may require the computer system 500 to be coupled to a host device. Several computing devices may be coupled to a single host device using the host device as a server. Alternatively or additionally, the computer system 500 may be coupled to several host devices (e.g., for each of the plurality of the host devices to serve as a backup for data stored in the computer system 500).

This written description uses examples to describe the subject matter herein, including the best mode, and also to enable any person skilled in the art to make and use the subject matter. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method for identifying inorganic matter during an ultrasound scan, the method comprising:
a) determining a tissue density threshold, wherein said tissue density threshold is a tissue density of a region of a body of said organism;
b) obtaining an ultrasound image of said region of said body of said organism;
c) identifying a first set of sub regions of said ultrasound image and a second set of sub regions of said ultrasound image based on said tissue density threshold, wherein said first set of sub regions pertains to organic matter and said second set of sub regions pertains to said inorganic matter; and
d) highlighting said second set of sub regions in said ultrasound image of said ultrasound scan.

2. The method as claimed in claim 1, wherein determining said tissue density threshold comprises:
a. receiving a sample ultrasound image of said region of said organism; and
b. calculating said tissue density threshold using said sample ultrasound image.

3. The method as claimed in claim 1, wherein determining said tissue density threshold comprises:
a. receiving a threshold value; and
b. setting said tissue density threshold in accordance with said threshold value.

4. The method as claimed in claim 1, wherein said method further comprises generating an alert based on a pre-defined condition.

5. The method as claimed in claim 1, wherein said method further comprises:
a. receiving a sub region information of a selected sub region in said ultrasound image;
b. calculating density of said selected sub region of said ultrasound image;
c. identifying at least one sub region based on said calculated density of said selected region; and
d. highlighting said at least one sub region.

6. The method as claimed in claim 1, wherein highlighting said identified second set of sub regions comprises drawing a boundary around each sub region of identified second set of sub regions.

7. The method as claimed in claim 1, wherein highlighting said identified second set of sub regions comprises coloring said identified second set of regions based on a color mode.

8. The method as claimed in claim 1, further comprising adjusting spectrum and intensity of highlighted second set of sub regions.

9. A method for highlighting inorganic matter during an ultrasound scan, the method comprising:
a) determining a tissue density threshold by using a sample ultrasound image of a region of a body, wherein said tissue density threshold is a tissue density of said region of said body of said organism;
b) capturing an ultrasound image of said region of said body of said organism;
c) identifying a first set of sub regions of said ultrasound image and a second set of sub regions of said ultrasound image based on tissue density threshold, wherein said first set of sub regions pertains to organic matter and said second set of sub regions pertains to said inorganic matter; and
d) highlighting said second set of sub regions based on a color mode in said ultrasound image of said ultrasound scan.

10. The method as claimed in claim 9, wherein determining said tissue density threshold comprises:
a. receiving a threshold value; and
b. setting said tissue density threshold in accordance with said threshold value.

11. The method as claimed in claim 9, wherein said method further comprises generating an alert based on a pre-defined condition.

12. The method as claimed in claim 9, wherein said method further comprises:
a. receiving a sub region information of a selected sub region in said ultrasound image;
b. calculating density of said selected sub region of said ultrasound image;
c. identifying at least one sub region based on said calculated density of said selected region; and
d. highlighting said at least one sub region.

13. The method as claimed in claim 9, wherein highlighting said identified second set of sub regions comprises drawing a boundary around each sub region of identified second set of sub regions.

14. A computer system for identifying inorganic matter during an ultrasound scan in an ultrasound imaging apparatus, the system comprising:
one or more processors; and
a non-transitory memory containing instructions that, when executed by said one or more processors, causes said one or more processors to perform a set of steps comprising:
a) determining, by a determination module of said one or more processors, a tissue density threshold, wherein said tissue density threshold is a tissue density of a region of a body of said organism;
b) obtaining, by a receiving module of said one or more processors, an ultrasound image of said region of said body of said organism;
c) identifying, by an identification module of said one or more processors, a first set of sub regions of said ultrasound image and a second set of sub regions of said ultrasound image based on said tissue density threshold, wherein said first set of sub regions pertains to organic matter and said second set of sub regions pertains to said inorganic matter; and
d) highlighting, by a highlighting module of said one or more processors, said second set of sub regions based on a color mode in said ultrasound image of said ultrasound scan.

15. The computer system as claimed in claim 14, wherein said determination module of said one or more processors is further adapted to:
a. receive a sample ultrasound image of said region of said organism; and
b. calculate said tissue density threshold using said sample ultrasound image.

16. The computer system as claimed in claim 14, wherein said determination module of said one or more processors is further adapted to:
a. receiving a threshold value; and
b. setting said tissue density threshold in accordance with said threshold value.

17. The computer system as claimed in claim 14, wherein said one or more processors is further configured to execute the steps of:
a. receiving a sub region information of a selected sub region in said ultrasound image;
b. calculating density of said selected sub region of said ultrasound image;
c. identifying at least one sub region based on said calculated density of said selected region; and
d. highlighting said at least one sub region.

18. The computer system as claimed in claim 14, wherein said highlighting module of said one or more processors is further adapted to draw a boundary around each sub region of identified second set of sub regions.

19. The computer system as claimed in claim 14, wherein said highlighting module of said one or more processors is further adapted to color the identified second set of regions based on a color mode.
